(19) United States
(12) Patent Application Publication
(10) Pub. No.: US 2024/0415995 A1
(43) Pub. Date: Dec. 19, 2024

(54) SYSTEM AND METHOD FOR RAPID AND STERILE TRANSFER OF A VIAL INTO AN ISOLATOR

(71) Applicant: COMECER S.P.A., Castel Bolognese (RA) (IT)

(72) Inventors: Alessandro Brunetti, Castel Bolognese (RA) (IT); Marco Testa, Castel Bolognese (RA) (IT)

(21) Appl. No.: 18/704,270

(22) PCT Filed: Oct. 28, 2022
(86) PCT No.: PCT/IB2022/060373

§ 371 Date: Apr. 24, 2024

(30) Foreign Application Priority Data
Oct. 28, 2021 (IT) 102021000027662
Oct. 28, 2021 (IT) 102021000027668

(51) Int. Cl. A61L 2/20; A61L 2/24; A61L 2/26; A61L 101/02

(52) U.S. Cl. CPC A61L 2/208; A61L 2/24; A61L 2/26; A61L 2101/02; A61L 2202/11; A61L 2202/121; A61L 2202/122; A61L 2202/14; A61L 2202/23

(57) ABSTRACT

A system for the rapid and sterile transfer of a vial into a pharmaceutical isolator having a working chamber, the system having: a first chamber having a first opening for insertion of the vial; a second chamber communicating with the first chamber through a second opening and having a third opening for the exit of the vial; a support structure enclosing the first and second chambers and mountable in a wall of the working chamber so that the first opening faces an outer environment and the third opening faces the working chamber; a first transfer component movable in the first chamber between two positions aligned to the first opening and, respectively, to the second opening; a second component movable in the second chamber between two positions aligned to the second opening and, respectively, to the third opening; a ventilation apparatus for generating an air flow in the first chamber; and a sterilisation apparatus for circulating vaporised hydrogen peroxide in the second chamber.

### CROSS-REFERENCE TO RELATED APPLICATIONS

This patent application is related to Italian Patent Applications No. 102021000027662 and No. 102021000027668 filed on Oct. 28, 2021, the entire disclosure of which is incorporated herein by reference.

### TECHNICAL FIELD

The present invention relates to a system and method for a rapid and sterile transfer of a vial into an isolator, specifically an isolator for pharmaceutical use.

In particular, the present invention may be advantageously, but not exclusively applied in the step of transferring vials or containers for cryogenic storage of biological material (cryovials) from an outer environment to a working chamber of a pharmaceutical isolator, to which the following description will make explicit reference without losing its generality.

### BACKGROUND

It is known to insert a cryovial into a pharmaceutical isolator by a procedure that requires several manual steps by an operator. Specifically, the operator must first decontaminate the cryovial under a laminar flow hood manually using a decontaminating agent, such as an alcohol-based solution, and then insert the cryovial into the isolator through a suitable small opening, commonly known as a mouse-hole, in a wall or hatch of the isolator.

Therefore, the aforesaid procedure for inserting a cryovial into an isolator is not repeatable, in that the level of decontamination of the cryovial is strongly affected by the behaviour of the operator. Moreover, the procedure, being time-consuming, is relatively slow.

### SUMMARY

The aim of the present invention is to realise a system for transferring a cryovial into an isolator and to provide a corresponding method for transferring a cryovial into an isolator that are capable of overcoming the drawbacks described above and, at the same time, are easy and cost-effective to manufacture.

In accordance with the present invention, they are provided a system for the rapid and sterile transfer of a vial into an isolator, in particular an isolator for pharmaceutical use, and a method for the rapid and sterile transfer of a vial into an isolator, according to what defined in the appended claims.

### BRIEF DESCRIPTION OF THE DRAWINGS

The present invention will now be described with reference to the accompanying drawings, which show a non-limiting embodiment thereof, wherein:

FIG. 1 shows a block diagram of the system of the present invention for the rapid and sterile transfer of a vial into an isolator, such system implementing the method of the present invention;

FIG. 2 shows, from a sectional view along a vertical plane of symmetry, a part of the system of FIG. 1 mounted on an outer wall of an isolator;

FIG. 3 shows an axonometric view of a component of the system part of FIG. 2; and

FIGS. 4 to 11 show the system part of FIG. 1 during as many operating steps.

### DESCRIPTION OF EMBODIMENTS

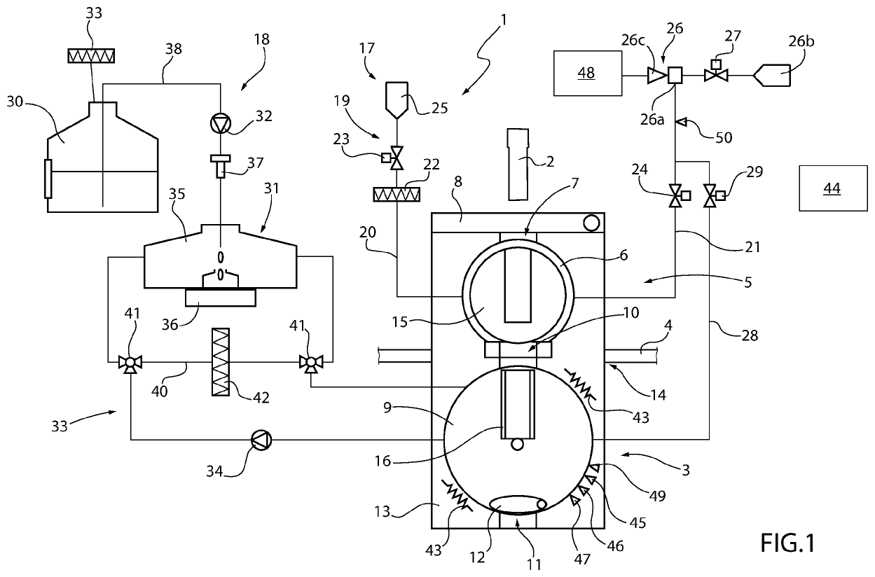

In FIG. 1, number 1 generically denotes a system for the rapid and sterile transfer of a vial 2 into an isolator, in particular an isolator for pharmaceutical use, which comprises a working chamber 3 having at least an outer wall 4 separating the working chamber 3 from an outer environment 5.

The transfer system 1 comprises a first chamber 6 having a first opening 7 provided with a first hatch 8 for inserting the vial 2 into the first chamber 6, a second chamber 9 communicating with the first chamber 6 through a second opening 10 and having a third opening 11 provided with a second hatch 12 for the exit of the vial 2, and a support structure 13, which encloses the two chambers 6 and 9 and that is mountable at a mounting opening 14 of the wall 4 in such a way that the opening 7 faces the outer environment 5 and the opening 11 faces the working chamber 3. Advantageously, the hatch 12 is motorised.

The transfer system 1 comprises a first transfer component 15, which is arranged in chamber 6, and a second transfer component 16, which is arranged in chamber 9. The transfer components 15 and 16 have respective shapes, shown in a simplified manner in FIG. 1, to accommodate a single vial 2. The transfer component 15 is movable between a first position, where it is aligned with the opening 7 to receive the vial 2 (FIG. 1), and a second position, wherein it is aligned with the opening 10 to transfer the vial 2 into the chamber 9. The second transfer component 16 is movable between a third position (FIG. 1), wherein it is aligned with the opening 10 to receive the vial 2, and a fourth position, wherein it is aligned with the opening 11 to transfer the vial 2 into the working chamber 3. Advantageously, the transfer components 15 and 16 are independently motorised.

The transfer system 1 further comprises a ventilation apparatus 17, which is connected to the chamber 6 to circulate in the latter an air flow satisfying a certain class of particle content, and a sterilisation apparatus 18, which is connected to the chamber 9 to circulate in the latter a gaseous sterilising fluid, preferably vaporised hydrogen peroxide. The particle content class is, for example, class B according to EC GMP Guide, Annex 1.

Still referring to FIG. 1, the ventilation apparatus 17 comprises a pneumatic circuit 19 which comprises an inlet branch 20 and an outlet branch 21 communicating with the chamber 7, a high-efficiency filter 22, e.g. a HEPA filter, and a modulating valve 23 in the inlet branch 20 and a further modulating valve 24 in the outlet branch 21.

In particular, the input branch 20 includes an input 25 that may be connected to a compressed air source (not shown). The modulating valve 23 is connected between the inlet 25 and the filter 22. The filter 22 has a pore size of less than 0.3 μm, in particular equal to 0.22 μm, to retain particles larger than the pores. Thereby, the inlet branch 20 is able to feed filtered compressed air with a variable flow rate into the chamber 6.

The pneumatic circuit 19 comprises an air extraction device 26, e.g. consisting of a Venturi ejector, connected at the end of the outlet branch 21 to draw air out of the chamber 6. The air extraction device 26 has an inlet 26a for a drawn fluid that is connected to the outlet branch 21 and an inlet 26b for a motor fluid. The inlet 26b may be connected to a compressed air source (not shown). The pneumatic circuit 19 comprises an on/off valve 27 connected to the inlet 26b to control the supply of compressed air to the air extraction device 26. The modulating valve 24, on the other hand, allows to adjust the flow rate of the air drawn from the chamber 6.

The pneumatic circuit 19 comprises an additional outlet branch 28, which consists in a duct connecting the chamber 9 with the air extraction device 26. The outlet branch 28 has an additional modulating valve 29 to adjust the flow rate of the air drawn from the chamber 9. The aim of the outlet branch 28 is to adjust the pressure inside the chamber 9, as will be further explained hereinafter.

The sterilisation apparatus 18 comprises a reservoir 30 to contain liquid hydrogen peroxide, a vaporizer 31 to vaporise hydrogen peroxide, a first pump 32 to feed the liquid hydrogen peroxide to the vaporizer 31, a pneumatic circuit 33 having a second pump 34 and connected to the chamber 9 to circulate the vaporised hydrogen peroxide in the latter.

In particular, the vaporizer 31 comprises a chamber 35 connected to the pneumatic circuit 33, a heater 36, in particular an electric heater, e.g. in the form of a plate, arranged in the chamber 35, and a needle 37, which protrudes into the chamber 35 above the heater 36 and is fed by the 15 pump 32 to drip the liquid hydroxide peroxide onto the heater 36.

The pump 32 has an inlet connected to a duct 38 which picks up from the tank 30. Advantageously, the pump 32 is a peristaltic pump. The reservoir 30 has a filter 39 to draw 20 in filtered air as a result of liquid hydrogen peroxide extraction. The filter 39 is a high efficiency filter, e.g. a HEPA filter. In particular, the filter 39 has a pore size of less than 0.3 μm, specifically equal to 0.22 μm.

The pneumatic circuit 33 comprises a bypass branch 40 connected in parallel to the vaporizer 31 via a pair of three-way valves 41 so that the vaporizer 31 can be excluded from an air circulation path flowing through the chamber 9. The bypass branch 40 comprises a filter 42 to allow removing the particulate matter during a step of aerating the chamber 9. The filter 42 is a high efficiency filter, e.g. a HEPA filter. In particular, the filter 42 has a pore size of less than 0.3 μm, in particular equal to 0.22 μm.

The pump 34 is connected between the chamber 9 and one of the two three-way valves 41 so as to be able to generate both the circulation of sterilising fluid, while transferring a vial 2 from the outer environment 5 to the working chamber 3 of the isolator, and the circulation of air, during the aforementioned step of aerating the chamber 9. Advantageously, the pump 34 is a diaphragm pump.

The transfer system 1 comprises a plurality of electrical heating elements 43 arranged in the support structure 13 to heat the chamber 9 in order to prevent condensation of the liquid hydrogen peroxide in the chamber 9 and thus transfer a dry vial 2 into the isolator.

The transfer system 1 comprises a plurality of sensors and a control unit 44 configured to control various motorised or electro-actuated components, in particular the hatch 12, the transfer components 15 and 16, the pumps 32 and 34, the modulating valves 23, 24, 27, 29 the three-way valves 41, the vaporizer 31 and the electrical heating elements 43, depending on one or more of the signals provided by the aforementioned sensors and on the basis of a sequence of steps involving the conditioning of the chambers 6 and 9 and the transfer of the vial 2 into the isolator.

In particular, the transfer system 1 comprises a temperature sensor 45 to measure the temperature in the chamber 9 and a humidity sensor 46 to measure the relative humidity in the chamber 9. The control unit 44 is configured to control the electrical heating elements 43 according to the signals provided by the temperature sensor 45 and humidity sensor 46 in such a way as to prevent condensation of the sterilising fluid in the chamber 9.

The transfer system 1 includes a pressure sensor 47 to measure the pressure in the chamber 9. The control unit 44 is configured to control the modulating valve 29 according to the signal provided by the pressure sensor 47 in order to maintain a constant pressure in the chamber 9. Indeed, the heating of the chamber 9 would cause, in the absence of the outlet branch 28, temporary increases in pressure within the chamber 9 during certain steps of the transfer process of the vial 2 during which the chamber 9 remains closed. The constant pressure, combined with temperature and relative humidity control, helps prevent condensation of the sterilising fluid.

The air extraction device 26 has its own outlet 26c connected to a neutralising device 48 adapted to neutralise the sterilising fluid arriving from the outlet 26c. The sterilising fluid arriving from the outlet 26c of the air extraction device 26 comes from the chamber 9 while the pressure therein is adjusted and, in small amounts, also from the chamber 6 through the opening 10 due to the transfer of the vial 2 from the chamber 6 to the chamber 9. In case the sterilising fluid is vaporised hydrogen peroxide, the neutralising device 48 comprises a catalyst to decompose the hydrogen peroxide into water.

According to alternative embodiments, the neutralising device 48 is either a part of the transfer system 1 or external to it.

The transfer system 1 comprises a sterilising fluid concentration sensor 49 to measure the concentration of the sterilising fluid in the chamber 9. Preferably, the sterilising fluid concentration sensor is arranged in the chamber 9. The control unit 44 is configured to control the pump 32 in such a way as to adjust the injection of liquid hydrogen peroxide into the vaporizer 31 at a desired value in terms of ml/min.

In the example shown in FIG. 1, the outlet branches 21 and 28, downstream of the respective modulating valves 24 and 29, converge in a single duct connected to the inlet 26a of the air extractor device 26. The transfer system 1 comprises an additional sterilising fluid concentration sensor 50 arranged in the aforementioned duct connected to the inlet 26a, and thus downstream of both modulating valves 21 and 24, to measure the sterilising fluid concentration reaching the air extraction device 26. This measurement is used by the control unit 44 to evaluate safety, i.e. to monitor the concentration of sterilising fluid being expelled from the transfer system 1.

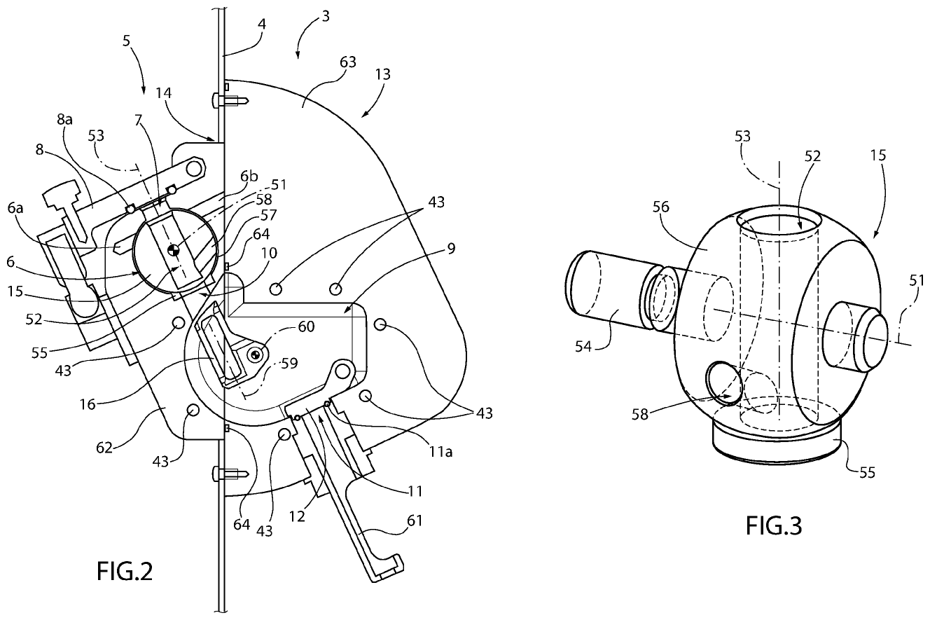

FIGS. 2 and 3 show a particular embodiment of the support structure 13, chambers 6 and 9 and transfer components 15 and 16. In particular, FIGS. 2 and 3 show the transfer component 15 in said first position (to receive the vial 2 from the outer environment 5) and the transfer component 16 in said third position (to receive the vial from the chamber 6).

Referring to FIGS. 2 and 3, the support structure 13 may be mounted at the mounting opening 14 in such a way that the opening 7 faces upwards, the chamber 9 is arranged downwards with respect to the chamber 6 and the opening 11 faces downwards, so that the transfer component 15 in the second position drops the vial 2 by gravity into the chamber 9 and the transfer component 16 in the fourth position drops the vial by gravity into the working chamber.

Hatch 8 and hatch 12 are normally closed. In FIG. 2, the hatches 8 and 12 are shown as closed. The hatch 8 has an annular-shaped gasket 8a adapted to contact an annular portion of the structure 13 surrounding the opening 7 for air-tightly closing the opening 7. The opening 11 has an annular-shaped gasket 11a adapted to contact the hatch 12 for air-tightly closing the opening 11.

The transfer component 15 is in the form of a rotational solid defined by rotation about an axis 51, perpendicular to the plane of the view of FIG. 2, and comprises a hole 52, which extends along an axis 53 perpendicular to axis 51 and is blind to serve as a housing for the vial 2. The transfer component 15 is motorised to rotate about the axis 51 between said first position, wherein the hole 52 is arranged facing the opening 7, and said second position, wherein the hole 52 is arranged facing the opening 10.

Advantageously, the transfer component 15 is in the form of a drum defined around the axis 51. In particular, the rotational solid defining the shape of the transfer component 15 is a sphere lacking two opposite spherical segments coaxial to the axis 51, as shown in FIG. 3. FIG. 3 also shows the drive shaft 54 of the motor that rotates the transfer component about the axis 51.

Advantageously, the transfer component 15 is made in a single piece.

The openings 7 and 10 are oriented according to respective axes (not shown) that are coaxial to axis 53. Preferably, the openings 7 and 10 are aligned with each other, i.e. their axes coincide.

The transfer system 1 comprises an annular hermetic sealing element 55, which is arranged around the opening 10 and has a cross-section shaped in such a way as to always remain in contact with a lateral outer surface 56 (FIG. 3) of the transfer component 15 to prevent the sterilising fluid from passing from the chamber 9 into the chamber 6 while loading the vial 2 into the transfer component 15 through the opening 7. In particular, the hermetic sealing element 55 avoids passages of sterilising fluid from the chamber 9 to the chamber 6 when the hole 52 does not communicate with the opening 10, for example when the transfer component 15 is in said first position (FIGS. 2 and 3) or in all the other angular positions except those in which the hole 52 is even partially in communication with the opening 10.

The chamber 6 comprises an inlet 6a and an outlet 6b (FIG. 2) which are connected with the pneumatic circuit 19 (not shown in FIG. 2) of the ventilation apparatus 17 for generating the air flow in the chamber 6. The chamber 6 has a shape similar to the outer shape of the transfer component 15 so as to define an air gap 57 of substantially uniform thickness between an inner surface of the chamber 6 and the lateral outer surface 56 of the transfer component 15. This aforesaid air flow circulates in the air gap 57 from the inlet 6a to the outlet 6b.

The transfer component 15 comprises an additional hole 58 transverse to the axis 53 and communicating with the hole 52. In particular, the hole 58 connects a bottom portion of the volume of the hole 52 with the air gap 57 to allow circulation of the air flow in the hole 52 and thus ease maintaining the class of particle content also in the hole 52 and remove any residual sterilising fluid from the hole 52 before opening the hatch 8 for a subsequent transfer cycle of another vial 2.

The transfer component 16 consists of a basket, which is shaped to accommodate the vial 2 oriented according to an axis 59, and is motorised to rotate about an axis 60 perpendicular to the plane of the view of FIG. 2, and in particular perpendicular to an ideal plane on which the axis 59 lies, and along a trajectory comprising said third position and said fourth position. The basket shape of the transfer component 16 offers a limited surface area in contact with the vial 2 so that, in use, the sterilising fluid in the chamber 9 can lap as much outer surface of the vial 2 as possible.

Advantageously, the transfer component 16 is made in a single piece.

The opening 11 has a guide 61 which protrudes outside the chamber 9, and thus, in use, inside the working chamber 3 of the isolator, to bring and hold the vial 2 in a pick-up position which is easily accessible by the gloves worn by an operator and by an automatic pick-up system arranged in the working chamber 3.

The support structure 13 is advantageously subdivided into a first support body 62 and a second support body 63, at least one of which is fixable to the wall 4 of the isolator, about the mounting opening 14, and which are hermetically fixed to each other with the interposition of an O-ring 64, through the mounting opening 14 so that, in use, the support body 62 is arranged in the outer environment 5 and the support body 63 is arranged in the working chamber 3 of the isolator.

In the example of FIG. 2, the support body 63 is fixed to the wall 4. Furthermore, the chamber 6 is defined in the support body 62 and the chamber 9 has a portion defined in the support body 62 and a remaining portion defined in the support body 63. The O-ring 64 is arranged about a connecting section between the two parts of the chamber 9.

FIG. 2 also shows the electric heating elements 43 arranged about the chamber 9. A given number of the electrical heating elements 43 are embedded in the support body 62 and the remaining number of the electric heating elements 43 are embedded in the support body 63.

The transfer system 1 may be employed to perform a method for a rapid and sterile transfer of a vial into an isolator, such a method comprising a plurality of steps described below referring in particular to the block diagram in FIG. 1 and FIGS. 4-11.

As already mentioned, the transfer system 1 is capable of transferring one vial 2 at a time into the working chamber 3 of the isolator. Normally, during a day of use of the isolator, a certain number of vials are transferred into the isolator.

Before transferring the vials, a step of pre-conditioning the chambers 6 and 9 is performed, in order to clean the air in the chambers 6 and 9, i.e. to achieve a certain class of particle content, and it is thus performed with the vaporizer 31 switched off.

The pre-conditioning step involves generating an air flow in the chamber 6 satisfying said class of particle content, by the pneumatic circuit 19, while the opening 7 is closed by the hatch 8 and the opening 10 is closed by the transfer component 15 in the first position (FIG. 2). For this purpose, the modulating valves 23 and 24 of the pneumatic circuit 19 and the valve 27 associated with the air extraction device 26 are open.

The pre-conditioning step also involves generating a circulation of air in the chamber 9 by the pump 34 of the pneumatic circuit 33 to achieve, in chamber 9 as well, said class of particle content. For this purpose, the three-way valves 41 are switched to exclude the vaporizer 31 from the air circulation and thus make air circulate through the bypass branch 40 and the modulating valve 29 is shut.

The air flow into the chamber 6, generated by feeding compressed air into the chamber 6 through the inlet branch 20 of the pneumatic circuit 19 and drawing air from the chamber 6 through the outlet branch 21 of the pneumatic circuit 19, is no longer interrupted in the subsequent steps of the method for transferring the vial 2. In other words, the modulating valves 23 and 24 of the pneumatic circuit 19 and the valve 27 associated with the air extraction device 26 remain open at all times.

After the pre-conditioning step, the conditioning of the chamber 9 is activated by circulating sterilising fluid. Specifically, the vaporizer 31 is activated to generate the sterilising fluid and the three-way valves 41 are switched to exclude the bypass branch 40 in order to maintain a circulation of sterilising fluid in the chamber 9 through the pneumatic circuit 33. The electrical heating elements 43 are switched on and controlled according to the temperature and relative humidity measured by the temperature sensor 45 and humidity sensor 46 to prevent condensation of the sterilising fluid. The modulating valve 29 is opened and adjusted according to the pressure measured by the pressure sensor 47 in order to maintain conditions in the chamber 9 suitable for preventing condensation of the sterilising fluid.

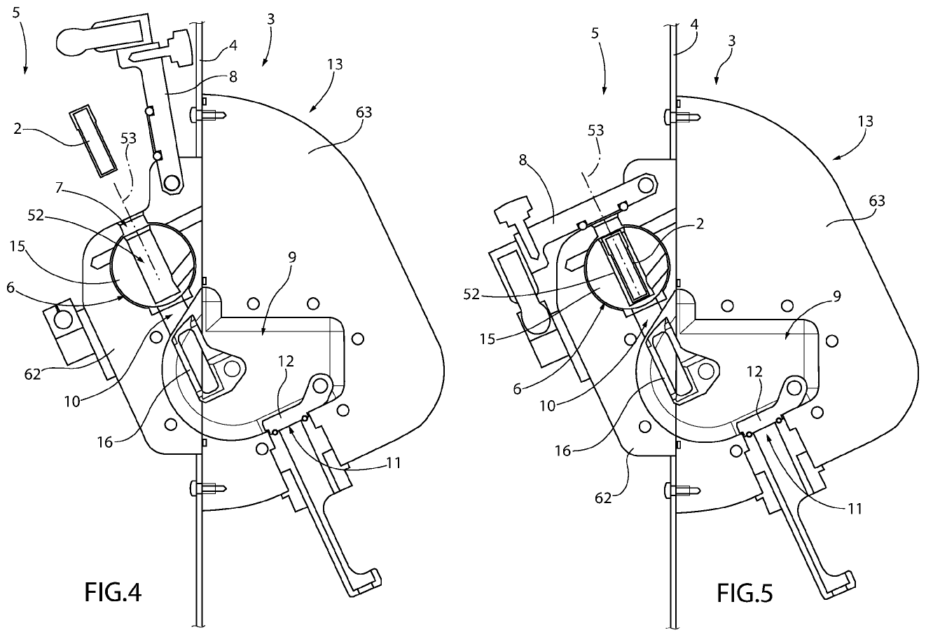

At this point, the transfer system 1 is ready to receive the vial 2 as shown in FIGS. 4 and 5. In particular, an operator temporarily opens the hatch 8 (FIG. 4) to insert the vial 2 into the chamber 6, where the vial 2 is accommodated in the hole 52 of the transfer component 15 in the first position (FIG. 5). Note that in the first position the transfer component 15 has the axis 53 of the hole 52 coinciding with the axis of the opening 7.

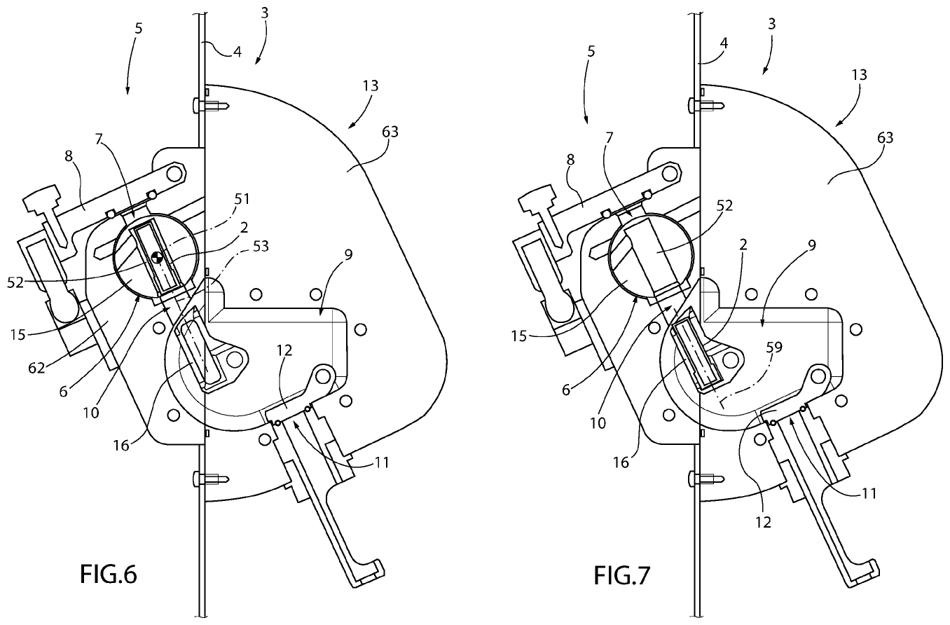

As a response to a command provided by the operator or generated by closing the hatch 8 by means of a button (not shown), preferably integrated in the support body 62, the transfer component 15 rotates about the axis 51 until it reaches the second position, thereby releasing the passage through the opening 10 (FIG. 6). In the second position, the axis 53 of the hole 52 coincides with the axis of the opening 10. In this position, the vial 2 drops by gravity into the chamber 9, leaving the hole 52 and passing through the opening 10. In chamber 9, the vial 2 is accommodated in the transfer component 16, which is located in the third position (FIG. 7). In the third position, the axis 59 of the transfer component 16 coincides with the axis of the opening 10.

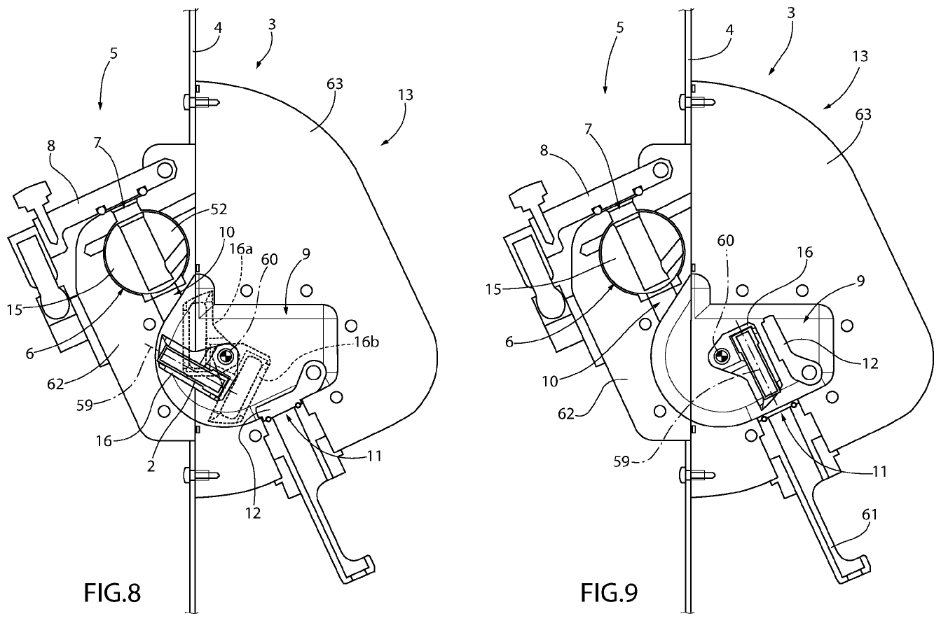

The transfer component 15 temporarily remains in the second position, in particular for a time interval that is predetermined by the time taken by the vial 2 to reach the chamber 9, after which the transfer component 15 returns to the first position (FIG. 8).

As mentioned above, the air flow in the chamber 6 is not interrupted. Thereby, the sterility conditions in the chamber 9 are substantially preserved and any residual sterilising fluid that might pass from the chamber 9 to the chamber 6 through the opening 10 during the transfer of vial 2 in the opposite direction would be drawn out of outlet branch 21 before opening the hatch 8 for a subsequent transfer cycle of another vial 2. The hole 58 facilitates the removal of sterilising fluid residues in the hole 52.

The vial 2 remains in the chamber 9 with the opening 10 closed by the transfer component 15 and the opening 11 closed by the hatch 12 for a predetermined time interval in order to allow the outer surface of the vial 2 to be sterilised. In this step, the transfer component 16 is moved one or more times between two positions such that the contact points between the vial 2 and the transfer component 16 vary to facilitate sterilisation of the entire outer surface of the vial 2. In particular, referring to FIG. 8, the transfer component 16 is rotated about the axis 60 between a fifth position, which is denoted by number 16a in FIG. 8 and lies beyond the trajectory between the third position (FIG. 7) and the fourth position (FIG. 9), and a sixth position, which is denoted by number 16a in FIG. 8 and lies between the third position and the fourth position. For example, in the fifth position, the transfer component 16 has its axis 59 arranged vertically.

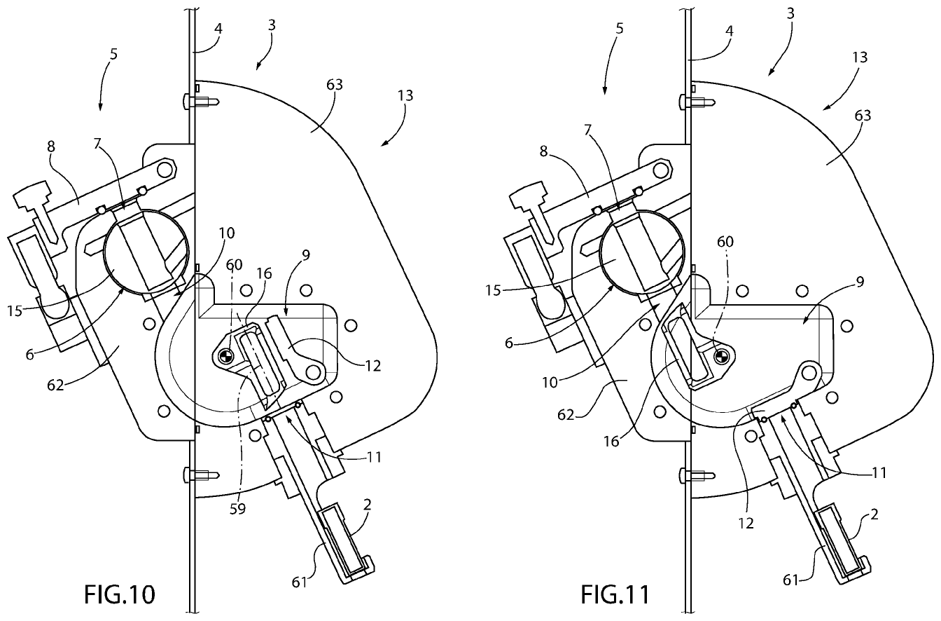

When the sterilisation step of the vial 2 is complete, the hatch 12 opens and the transfer component 16 rotates to the fourth position (FIG. 9). In the fourth position, the axis 59 of the transfer component 16 coincides with the axis of the opening 11. In such position, the vial 2 drops by gravity into the guide 61, leaving the transfer component 16 and passing through the opening 11 (FIG. 10).

The air drawn from the chamber 9 through the outlet branch 28 of the pneumatic circuit 19 prevents any small amounts of sterilising fluid from leaving the chamber 9 and entering the working chamber 3.

At this point, the transfer component 16 is returned to the third position and the hatch 12 is closed (FIG. 11). The transfer system 1 is thus ready to receive another vial 2 to be transferred to the working chamber 3, i.e. to perform the transfer cycle again for another vial 2.

Advantageously, the generation of the air flow into the chamber 6 occurs by adjusting the modulating valves 23 and 24 in such a way that the flow rate of the air drawn from the chamber 6 differs from the flow rate of the filtered compressed air fed into the chamber 6 as the steps of the transfer cycle of the vial 2 change.

For example, when the transfer component 15 is in the first position (FIGS. 4, 5, 8-11), the valves 23 and 24 are adjusted in such a way that the flow rate of the air fed into chamber 6 is greater than the flow rate of the air drawn from the chamber 6 so that when the hatch 8 is opened again to receive a subsequent vial 2, the air from the outer environment 5 does not enter the chamber 6, thus reducing the risk of contamination of the chamber 6.

The method for the rapid and sterile transfer of a vial 2 also involves a step of globally aerating the transfer system 1 while keeping the vaporizer 31 switched off, i.e. of the two chambers 6 and 9 and of chamber 35 of the vaporizer 31, which may be performed, for example, at the end of a day of use of the isolator.

The step of globally aerating also involves circulating, in addition to the usual air flow into the chamber 6 through the inlet branch 20 and outlet branch 21 with the hatch 8 closed and opening 10 closed, an additional air flow into the chambers 9 and 35 through the pneumatic circuit 33 and outlet branch 28 with the hatch 12 open. Thus, the modulating valves 23, 24 and 29 are open and the three-way valves 41 are switched so as to exclude the bypass branch 40. Thereby, the class of particle content in the chamber 6 is maintained while any residual sterilising fluid is removed from the chambers 9 and 35 and is expelled from the air extractor device 26.

According to a further embodiment not shown, the transfer system 1 differs from the one shown in the Figures and described above in that it comprises, instead of the chambers 6 and 9 and the rotating-type transfer components 15 and 16, two chambers extending according to two respective directions parallel to each other, and two transfer components of the translating type, each of which is in the form of a cylinder without bases extending along its own longitudinal axis, is arranged in a respective chamber with its longitudinal axis perpendicular to the direction of the chamber and is adapted to translate into the chamber along that direction. Thus, a first one of the two transfer components translates in a first one of the two chambers between a first position, in which it is aligned to a first opening of the first chamber to receive the vial, and a second position, in which it is aligned to a second opening that connects the two chambers to transfer the vial into the second chamber; the second one of the two transfer components translates into the second one of the two chambers between a third position, in which it is aligned with a second opening to receive the vial, and a fourth position, in which it is aligned with a third opening of the second chamber to transfer the vial into the working chamber.

Although the above-described invention specifically refers to a very specific embodiment, it is not to be intended as limited to that embodiment, being included in its scope all those variations, modifications or simplifications as covered by the appended claims, such as, for example:
the transfer component 15 has the shape of a different rotational solid, for example a cylinder coaxial to the axis 51, and the sealing element 55 is shaped accordingly; and
the support body 62 is fixable to the isolator wall 4 and the support body 63 is sealingly fixed to the support body 63.

The main advantage of the above-described transfer system 1 and of the method for the rapid and sterile manner transfer of a vial into an isolator is that it allows the rapid insertion of a vial 2 into a working chamber 3 of an isolator without having to follow a manual procedure for decontaminating the vial 2. More specific advantages result clearly from the particular characteristics of the transfer system 1 and the particular steps of the method as described above. For example, the chambers 6 and 9 communicating only through the opening 10, the transfer components 15 and 16 movable inside the respective chambers 6 and 9 and the ventilation apparatus 17 connected to the chamber 6 make it possible to isolate the chamber 9, in which the sterilisation of the vial 2 takes place, from the outer environment 5 and at the same time from the working chamber 3 of the isolator. Another advantage is that the vial 2 transferred into the chamber 3 retains the same position as when it is Loaded into the chamber 6.

1. A system for rapid and sterile transfer of a vial into an isolator, in particular an isolator for pharmaceutical use, comprising a working chamber (3) having at least one outer wall (4) separating the working chamber (3) from an outer environment (5), the system (1) comprising: a first chamber (6) having a first opening (7) provided with a first hatch (8) for insertion of the vial (2) into the first chamber (6); a second chamber (9) communicating with the first chamber (6) through a second opening (10) and having a third opening (11) provided with a second hatch (12) for exit of the vial (2); a support structure (13), which encloses first and second chambers (6, 9) and is mountable at a mounting opening (14) of the outer wall (4) in such a way that the first opening (7) faces the outer environment (5) and the third opening (11) faces the working chamber (3); a first transfer component (15) arranged in the first chamber (6) and movable between a first position, in which it is aligned with the first opening (7) to receive the vial (2), and a second position, in which it is aligned with the second opening (10) to transfer the vial (2) into the second chamber (9); a second transfer component (16) arranged in the second chamber (9) and movable between a third position, in which it is aligned with the second opening (10) to receive the vial (2), and a fourth position, in which it is aligned with the third opening (11) to transfer the vial (2) into the working chamber (3); a ventilation apparatus (17) connected to the first chamber (6) to circulate in the latter an air flow satisfying a certain class of particle content; and a sterilization apparatus (18) connected to the second chamber (9) to circulate in the latter a gaseous sterilizing fluid, preferably vaporized hydrogen peroxide.

2. The system according to claim 1, wherein said support structure (13) is mountable at said mounting aperture (14) such that the first aperture (7) faces upwards, the second chamber (9) is arranged downwards relative to the first chamber (6) and the third aperture (11) faces downwards, so that the first transfer component (15) in the second position drops the vial (2) by gravity into the second chamber (9) and the second transfer component (16) in the fourth position drops the vial (2) by gravity into the working chamber (3).

3. The system according to claim 1, wherein in at least said first position the first transfer component (15) closes the second opening (10).

4. The system according to claim 1, wherein said first transfer component (15) is in the form of a rotational solid defined by rotation about a first axis (51), comprises a first hole (52), which extends along a second axis (53) perpendicular to the first axis (51) and is blind to serve as a housing for the vial (2), and is motorized to rotate about the first axis (51) between said first position, wherein the first hole (52) is faces the first opening (7), and said second position, wherein the first hole (52) faces the second opening (10).

5. The system according to claim 4, and comprising an annular hermetic sealing element (55) arranged around the second opening (10) and having a cross-section shaped in such a way as to remain in contact with a lateral outer surface (56) of the first transfer component (15) to prevent the sterilising fluid from passing from the second chamber (9) to the first chamber (6) when the first hole (52) does not communicate with the second opening (10).

6. The system according to claim 4, wherein said rotational solid is a sphere lacking two opposite spherical segments coaxial to the first axis (51).

7. The system according to claim 1, wherein said second transfer component (16) is a basket, which is shaped to accommodate the vial (2) in accordance with a third axis (59), and is motorised to rotate about a fourth axis (60) perpendicular to an ideal plane on which said third axis (59) lies and along a trajectory comprising said third position and said fourth position.

8. The system according to claim 1, wherein said sterilization apparatus (18) comprises a reservoir (30) for containing liquid hydrogen peroxide, an hydrogen peroxide vaporizer (31), a first pump (32) for feeding liquid hydrogen peroxide to the hydrogen peroxide vaporizer (31), and a first pneumatic circuit (33) provided with a second pump (34) and connected to the second chamber (9) for circulating the vaporized hydrogen peroxide therein.

9. The system according to claim 1, and comprising electrical heating elements (43) arranged in the support structure (13) so as to heat the second chamber (9), a temperature sensor (45) and a humidity sensor (46) for measuring the temperature and relative humidity in the second chamber (9), and a control unit (44) configured to control the electrical heating elements (43) according to signals from the temperature sensor (45) and humidity sensor (46) so as to prevent condensation of the sterilising fluid in the second chamber (9).

10. The system according to claim 1, wherein said ventilation apparatus (17) comprises a second pneumatic circuit (19), which comprises an inlet branch (20) and an outlet branch (21) communicating with the first chamber (6), a filter (22) and a first modulating valve (23) in the inlet branch (20) and a second modulating valve (24) in the outlet branch (21).

11. The system according to claim 4, wherein the first chamber (6) has a shape similar to the outer shape of the first transfer component (15) to define an air gap (57) of substantially uniform thickness between an inner surface of the first chamber (6) and an outer surface (56) of the first transfer component (15).

12. The system according to claim 11, wherein said first transfer component (15) comprises a second hole (58), which connects a bottom portion of the volume of said first hole (52) with said air gap (57) to allow circulation of said air flow in said first hole (52).

13. The system according to claim 1, wherein said second pneumatic circuit (19) comprises a further outlet branch (28) communicating with said second chamber (9) and provided with a third modulating valve (29); said system (1) comprising a pressure sensor (47) for measuring the pressure in said second chamber (9) and a control unit (44) configured to control said third modulating valve (29) according to the signal provided by said pressure sensor (47) so as to maintain a constant pressure in said second chamber (9).

14. The system according to claim 1, wherein said support structure (13) is divided into a first support body (62) and a second support body (63), at least one of which is fixable to said outer wall (4) and which are hermetically fixed to each other in such a way that, in use, the first support body (62) is arranged in the outer environment (5) and the second support body (63) is arranged in the working chamber (3) of the isolator; preferably, said first chamber (6) being defined in the first support body (62) and said second chamber (9) being defined at least partially in the second support body (63).

15. An isolator, in particular for pharmaceutical use, comprising a working chamber (3), which has at least one outer wall (4) separating said working chamber (3) from an outer environment (5) and presenting a mounting opening (14), and a system (1) for rapid and sterile transfer of a vial (2) into said working chamber (3), said system (1) being according to claim 1 and said support structure (13) being mounted in correspondence with said mounting opening (14).

16. A method for a rapid and sterile transfer of a vial into an isolator,
in particular an isolator for pharmaceutical use, comprising a working chamber (3) having at least one wall (4) separating the working chamber (3) from an external environment (5), the method comprising:
generating in a first chamber (6) integral with the wall (4) an air flow satisfying a certain class of particle content, by means of a first pneumatic circuit (19) which feeds filtered compressed air into the first chamber (6) and sucks air from the first chamber (6), the first chamber (6) having a first opening (7) communicating with the external environment (5) and provided with a normally closed first hatch (8);
generating and maintaining in circulation in a second chamber (9) integral with the wall (4) a gaseous sterilizing fluid, by means of a second pneumatic circuit (33), the second chamber (9) having a second opening (11) communicating with the working chamber (3) and provided with a normally closed second hatch (12);
temporarily opening the first hatch (8) to insert the vial (2) into the first chamber (6) without interrupting the generation of the air flow;
receiving the vial (2) in the first chamber (6) by means of a first component (15), which is movable in the first chamber (6) and, when it receives the vial (2), closes a third opening (10) which puts in communication the first and second chambers (6, 9);
transferring the vial (2) from the first chamber (6) to the second chamber (9) through the third opening (10) following a movement of the first component (15) which temporarily releases the third opening (10); and
temporarily opening the second hatch (12) to transfer the vial (2) from the second chamber (9) to the working chamber (3).

17. The method according to claim 16, and comprising:
receiving the vial (2) in the second chamber (9) via a second component (16) which is movable in the second chamber (9);
the transfer of the vial (2) from the second chamber (9) to the working chamber (3) occurring by means of a movement of the second component (16).

18. The method according to claim 17, and comprising:
leaving the vial (2) in the second chamber (9) with the third opening (10) closed by the first component (15) and the second opening (11) closed by the second hatch (12) for a predetermined time interval in order to sterilise the outer surface of the vial (2); and
while the vial (2) is left in the second chamber (9), move the second component (16) in such a way as to vary contact points between the vial (2) and the second component (16) to facilitate sterilisation of the entire outer surface of the vial (2).

19. The method according to claim 16, wherein the filtered compressed air is fed in the first chamber (6) with a flow rate different from that with which the air is sucked from the first chamber (6).

20. The method according to claim 16, wherein generating in a first chamber (6) integral with the wall (4) an air flow comprises:
when the vial (2) is received in the first chamber (6) by means of said first component (15), feeding filtered compressed air into the first chamber (6) with a flow rate greater than that with which the air is drawn from the first chamber (6) to prevent air from the external environment (5) from entering the first chamber (6).

21. The method according to claim 16, and comprising:
measuring the pressure in the second chamber (9) by means of a pressure sensor (47); and
drawing air from the second chamber (9) through a pneumatic duct (28) and in a controlled manner based on the measured pressure so as to maintain a constant pressure in the second chamber (9).

22. The method according to claim 16, comprising:
measuring the temperature and relative humidity in the second chamber (9) by means of a temperature sensor (45) and a humidity sensor (46);
adjusting the temperature in the second chamber (9) by controlling activation of electrical heating elements (43) arranged around the second chamber (9) based on the measured temperature and the measured relative humidity in such a way as to prevent condensation of the sterilising fluid in the second chamber (9).

23. The method according to claim 16, wherein said sterilising fluid is vaporised hydrogen peroxide.
